(19) United States
(12) Patent Application Publication
(10) Pub. No.: US 2022/0347357 A1
(43) Pub. Date: Nov. 3, 2022

(54) COATINGS FOR TREATMENT DEVICE

(71) Applicant: OLYMPUS MEDICAL SYSTEMS CORP., Tokyo (JP)

(72) Inventor: Tsunetaka AKAGANE, Tokyo (JP)

(73) Assignee: OLYMPUS MEDICAL SYSTEMS CORP., Tokyo (JP)

(21) Appl. No.: 17/684,610

(22) Filed: Mar. 2, 2022

Related U.S. Application Data
(60) Provisional application No. 63/180,820, filed on Apr. 28, 2021.

(51) Int. Cl. A61L 31/08; A61B 18/14

(52) U.S. Cl. CPC A61L 31/088; A61B 18/1482; A61B 2018/00178

(57) ABSTRACT

A treatment device equipped with high frequency capability includes a pair of clasping jaws that serves as bipolar electrodes. Another treatment device equipped with high frequency capability includes a high-frequency knife that serves as monopolar electrode. An anti-fouling coating that minimizes or prevents adhesion of cauterized tissues is incorporated on the electrode surfaces. Durability and functioning of the anti-fouling coating is improved by one or a combination of increased coating thickness, increased density of the coating, and incorporation of glass and/or high molecular weight PFPE into the coating. The anti-fouling coating can be applied to different locations of the treatment portion, e.g., an insulated portion and a recess of an electrode, an insulated portion and a boundary portion with an electrode.

### RELATED APPLICATION DATA

The application is based on and claims priority under 35 U.S.C. § 119 to U.S. Provisional Application No. 63/180,820 filed on Apr. 28, 2021, the entire contents of which are incorporated herein by reference.

### FIELD OF DISCLOSURE

The present disclosure relates to a treatment device for high-frequency current procedure having a treatment portion that incorporates an anti-fouling coating on the electrode surfaces, either a monopolar electrode or bipolar electrodes, and methods of manufacturing such anti-fouling coatings.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

FIG. 11 is a figure of a treatment device disclosed in the related art (International Patent Application Publication No. WO2019/123606A1). FIG. 11 shows a cross-sectional view of an end effector assembly for use with an electrosurgical instrument that has a gripping jaw comprising a first gripping jaw 13 and a second gripping jaw 14. The first gripping jaw 13, which is a vibration transmission member 8 for transmitting ultrasonic vibration, serve as an electrode for transmitting high frequency currents, consists of a treatment surface 17, a back surface 19, and side surfaces 20. The treatment surface 17 of the first gripping jaw 13 is coated with an organic layer 41 and anti-stick coating 51, which are both monomolecular film layers. The organic layer 41 and anti-stick coating 51 extends over the treatment surface 17 and half of each side surface 20. The second gripping jaw 14 also includes a treatment surface 18, which is supported by the supporting member 21, and a short circuit prevention member 23. The related art describes organic layer 41 and anti-stick coating 51 covering the upper half of the gripping jaw 13, but fails to describe the ways the organic layer 41 and anti-stick coating 51 should be applied to the gripping jaws 13, 14 in order to improve the durability of the coating and/or improve the anti-stick characteristics of the coatings.

FIG. 12 is a figure of a treatment device disclosed in the related art (U.S. Pat. Publ. No. 2017/0119456A1). FIG. 12 shows a cross-sectional view of a jaw member of an end effector assembly for use with an electrosurgical instrument. The jaw member 100 consists of sealing plate 102 having a stainless steel layer 104 and an electrically insulative layer 106. Sealing plate 102 is affixed to support base 110, which is secured to insulative housing 112. A CrN coating 120 is disposed over the outer surface 114 of the assembled sealing plate 102, support base 110, and insulative housing 112. Additionally, an HMDSO plasma coating 130 is disposed over the CrN coating 120. The related art describes only partially coating the outer surface 114 of the jaw member or including thicker layers of the CrN coating 120 and/or the HMDSO plasma coating 130 on different portions of the outer surface 114 of the jaw member. However, the related art fails to prescribe which portions of the jaw member should be coated or applied thicker layers, which is essential information in terms of improving the functions of the coatings applied to the treatment device.

### SUMMARY

Accordingly, there is a need for designing a treatment device with an improved coating configuration to increase the durability and/or anti-fouling characteristics of the coating materials, which would substantially obviate one or more of the issues due to limitations and disadvantages of related art treatment device.

An object of the present disclosure is to provide an improved treatment device that provides an efficient design for the improving the durability and/or anti-fouling characteristics of the coating materials applied to the jaw members compared to the related art. At least one or some of the objectives is achieved by the treatment device disclosed herein.

Embodiments of the disclosed treatment device comprises a treatment device with a body including a connection configured to connect to a power source to supply power for conducting a high-frequency treatment with the treatment device and a longitudinally extending shaft having a proximal end connected to the body and a distal end defining a treatment end. The treatment end includes a first bipolar electrode and a second bipolar electrode for conducting high-frequency currents including a conductive treatment surface and an insulating cover, and the treatment end is coated with an anti-fouling coating including an insulating substance and a durability amplified coating.

Embodiments of the disclosed treatment device further comprises a treatment device with a body including a connection configured to connect to a power source to supply power for conducting a high-frequency treatment with the treatment device and a longitudinally extending shaft having a proximal end connected to the body and a distal end defining a treatment end. The treatment end includes a first bipolar electrode and a second bipolar electrode for conducting high-frequency currents including a conductive treatment surface and an insulating cover, and the treatment end is coated with an anti-fouling coating including an insulating substance and an anti-fouling amplified coating.

Embodiments of the disclosed treatment device further comprises a treatment device with a body including a movable slider and a connection configured to connect to a power source supplying power for conducting a high-frequency treatment with the treatment device, a longitudinally extending tube having a proximal end connected to the body, and a treatment end including monopolar electrode conducting high-frequency currents, an insulating chip, and a dissecting knife. The treatment end is coated with anti-fouling coating including an insulating substance and an anti-fouling strengthened portion.

Embodiments of the disclosed treatment device further comprises a treatment device with the conductive treatment surface of the first bipolar electrode including an insulation pad that is coated with durability amplified coating.

Embodiments of the disclosed treatment device further comprises a treatment device with the conductive treatment surface of the second bipolar electrode including an insulation pad that is coated with durability amplified coating.

Embodiments of the disclosed treatment device further comprises a treatment device with the conductive treatment surface of the second bipolar electrode that contacts the insulation pad of the first bipolar electrode when the first and second bipolar electrodes are closed together is coated with durability amplified coating.

Embodiments of the disclosed treatment device further comprises a treatment device with the treatment surface of first bipolar electrode including multiple notches with dents coated with anti-fouling amplified coating.

Embodiments of the disclosed treatment device further comprises a treatment device with the first bipolar electrode includes a cutting blade channel that is coated with anti-fouling coating.

Embodiments of the disclosed treatment device further comprises a treatment device with the anti-fouling amplified coating is applied to the boundary area of the conductive treatment surface and the insulating cover.

Embodiments of the disclosed treatment device further comprises a treatment device with the anti-fouling amplified coating is applied to the boundary area of the monopolar electrode and the insulating chip.

Embodiments of the disclosed treatment device further comprises a treatment device with the anti-fouling amplified coating is applied to the boundary area of the monopolar electrode and the dissecting knife.

Embodiments of the disclosed treatment device further comprises a treatment device with the anti-fouling coating including PFPE.

Embodiments of the disclosed treatment device further comprises a treatment device with the anti-fouling coating including glass.

Embodiments of the disclosed treatment device further comprises a treatment device with the thickness of the anti-fouling coating being 1 μm or less.

Embodiments of the disclosed treatment device further comprises a treatment device with the average thickness of the durability amplified coating being larger than the average thickness of the anti-fouling coating.

Embodiments of the disclosed treatment device further comprises a treatment device with the surface of the treatment end that the durability amplified coating is applied to being rougher compared to the surface of the treatment end the anti-fouling coating is applied to.

Embodiments of the disclosed treatment device further comprises a treatment device with the molecular weight of the insulating substance in the anti-fouling coating being different compared to the durability amplified coating or the anti-fouling coating.

Embodiments of the disclosed treatment device further comprises a treatment device with the molecular weight of the insulating substance in the anti-fouling coating being lower than the molecular weight of the insulating substance in the anti-fouling amplified coating.

Embodiments of the disclosed treatment device further comprises a treatment device with the molecular weight of the insulating substance in the anti-fouling coating being higher than the molecular weight of the insulating substance in the durability amplified coating.

Embodiments of the disclosed treatment device further comprises a treatment device with the molecular weight of the insulating substance in the anti-fouling amplified coating being higher than the durability amplified coating.

Embodiments of the disclosed treatment device further comprises a treatment device with the insulating substance the molecular weight being compared is PFPE.

Embodiments of the disclosed treatment device further comprises a treatment device with the base of the durability amplified coating configured to have higher density of polar functional groups compared to the anti-fouling coating.

Embodiments of the disclosed treatment device further comprises a treatment device with the base of the anti-fouling amplified coating configured to have higher density of polar functional groups compared to the anti-fouling coating.

Embodiments of the disclosed treatment device further comprises a treatment device with the frequency of bonding between the insulating substance and the base material on the surface of the treatment end being higher at the durability amplified coating compared to the anti-fouling coating.

Embodiments of the disclosed treatment device further comprises a treatment device with the frequency of bonding between the insulating substance and the base material on the surface of the treatment end being higher at the anti-fouling amplified coating compared to the anti-fouling coating.

Embodiments of the disclosed treatment device further comprises a treatment device with a glass coating base being formed between the surface of the treatment end and the durability amplified coating.

Embodiments of the disclosed treatment device further comprises a treatment device with a glass coating base being formed between the surface of the treatment end and the anti-fouling amplified coating.

Embodiments of the disclosed treatment device further comprises a treatment device with the durability amplified coating being bonded to the hydroxyl group formed on the glass coating base.

Embodiments of the disclosed treatment device further comprises a treatment device with the anti-fouling amplified coating being bonded to the hydroxyl group formed on the glass coating base.

Embodiments of the disclosed treatment device further comprises a treatment device with the insulating cover or the insulating chip including an antifouling substance that is less likely to bond with the anti-fouling coating compared to the other substances of the insulating cover or the insulation chip.

Embodiments of the disclosed treatment device further comprises a treatment device with the insulating cover or the insulating chip including a portion not coated with anti-fouling coating.

Embodiments of the disclosed treatment device further comprises a treatment device with the insulating cover placed opposite to the conductive treatment surface including a portion not coated with anti-fouling coating.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory and are intended to provide further explanation of the disclosed input device as claimed.

### BRIEF DESCRIPTION OF THE DRAWING

The following detailed description of preferred embodiments can be read in connection with the accompanying drawings in which like numerals designate like elements and in which:

FIG. 1 illustrates a treatment device including a body, a shaft, and a treatment end.

FIG. 2 illustrates a magnified view of the treatment end shown in FIG. 1 and showing the upper and lower jaws serving as bipolar electrodes in an open configuration.

FIG. 3 illustrates the chemical formula of the anti-fouling coating.

FIGS. 4A and 4B schematically illustrate an anti-fouling coating formed with glass coating.

FIG. 5 illustrates a cross-sectional view of the lower jaw and various amplification target areas.

FIG. 6 illustrates a treatment device including an operation portion, tube, insertion tube, and high-frequency knife.

FIG. 7 illustrates a magnified view of the high-frequency knife and various amplification target areas.

FIG. 8 illustrates the relationship between molecular weight of PFPE and durability and anti-fouling properties.

FIG. 9 is a graph illustrating the distribution histogram of anti-fouling coating including PFPE with varying molecular weights.

FIG. 10A illustrates the micrometer-scale morphology of a traditional composite material of PTFE and resin.

FIG. 10B illustrates the nanometer-scale morphology of advanced composite material of PTFE and resin.

FIGS. 11 and 12 are figures of a treatment device disclosed in the related art.

Throughout all of the drawings, dimensions of respective constituent elements are appropriately adjusted for clarity. For ease of viewing, in some instances only some of the named features in the figures are labeled with reference numerals.

### DETAILED DESCRIPTION

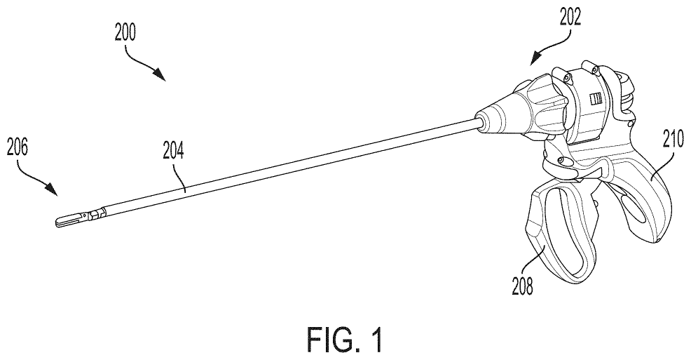

FIG. 1 is an illustration of a treatment device 200 including a body 202, a shaft 204, and a treatment end 206. The body 202 includes a moving arm 208 and a grip 210. The moving arm 208 is used to actuate and operate the functions of treatment end 206. The grip 210 is connected to a power source supplying power used for high-frequency treatment procedure by treatment device 200. The power source can be a wired or wireless power source. The shaft 204 protects the wires and members within, necessary for operating the functions of treatment end 206.

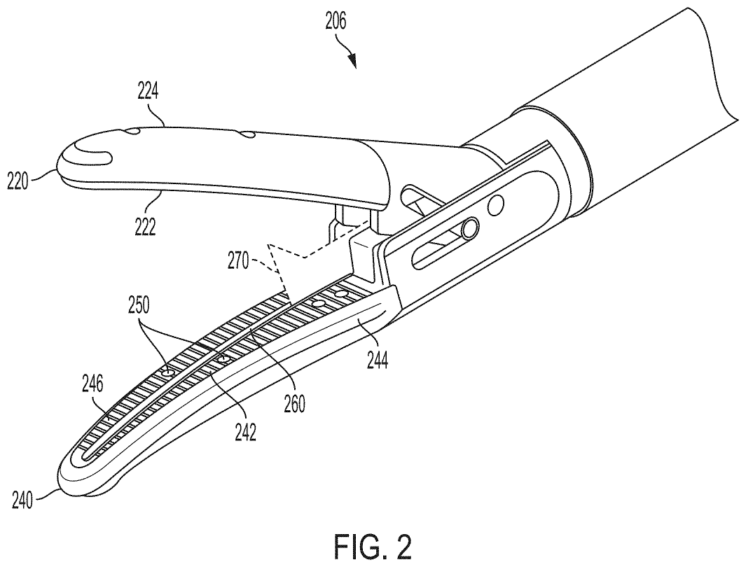

FIG. 2 is an illustration of the treatment end 206 including the upper jaw 220 and lower jaw 240. The upper jaw 220 and lower jaw 240 may serve as bipolar electrodes electrically conducting the power supplied from the body 202 through the shaft 204. When the upper jaw 220 and lower jaw 240 are in a closed position, high frequency currents would be conducted between the jaws, and any object clasped between the jaws would either be dissected or sealed using the conducted high-frequency currents.

Both the upper jaw 220 and lower jaw 240 consists of a treatment surface and insulation covers. Upper jaw 220 includes upper treatment surface 222 and upper insulation cover 224, and lower jaw 240 includes lower treatment surface 242 and lower insulation cover 244. The treatment surfaces are made by conductive materials such as metal to serve as bipolar electrodes conducting high frequency currents and includes multiple notches 246 for securely holding grasped objects. The insulation covers are made of non-conductive materials such as rubber and resin to insulate the high frequency currents flowing through the upper treatment surface 222 and lower treatment surface 242. The lower jaw 240 also includes insulation pads 250 for preventing short circuits to occur in case the treatment surfaces of the upper jaw 220 and lower jaw 240 are in a closed position and high frequency currents flow without any tissues or objects caught between the two jaws. The lower jaw 240 also includes a channel 260 configured to accommodate movement, such as sliding movement, of cutting blade 270 for cutting the tissue(s) grasped between the upper and lower jaws. In some embodiments, cutting blade 270 may also serve as a high frequency electrode. The upper jaw 220 may or may not have insulation pads on the upper treatment surface 222, and in case there was no insulation pads, the upper treatment surface 222 comes in direct contact with the insulation pads 250 of the lower jaw 240.

At least portions of the opposing surfaces of the upper jaw 220 and lower jaw 504 are coated with anti-fouling coating materials for preventing tissues cauterized through high frequency currents to stick to the treatment surfaces 222, 242 and other portions of the upper jaw 220 and lower jaw 240. The coating materials that may be used for the anti-fouling coating includes glass coating and monomolecular coating using perfluoropolyether (PFPE) or a functionalized PFPE as main agent, which together with glass coating form a thin insulating film having anti-fouling properties. The glass coating may have a cellular structure, in some embodiments the cellular structure has a brick wall structure of adjacent rows of offset block structures, which provides improved impact resistance in addition to anti-fouling effects.

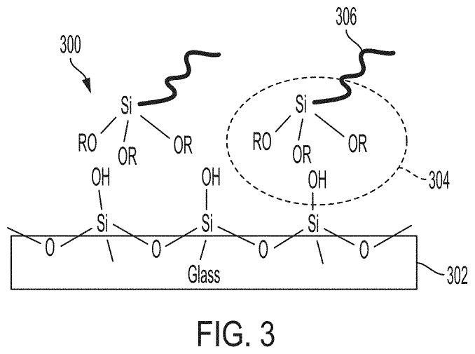

FIG. 3 is an illustration of the anti-fouling coating 300 formed with a monomolecular coating using PFPE as a main agent formed on a glass coating 302 using a silane coupling 304 (in alternative embodiments, a phosphate coupling may be used). The fluorine chain 306 is connected to the glass coating 302 using a silane coupling 304 to achieve anti-fouling property.

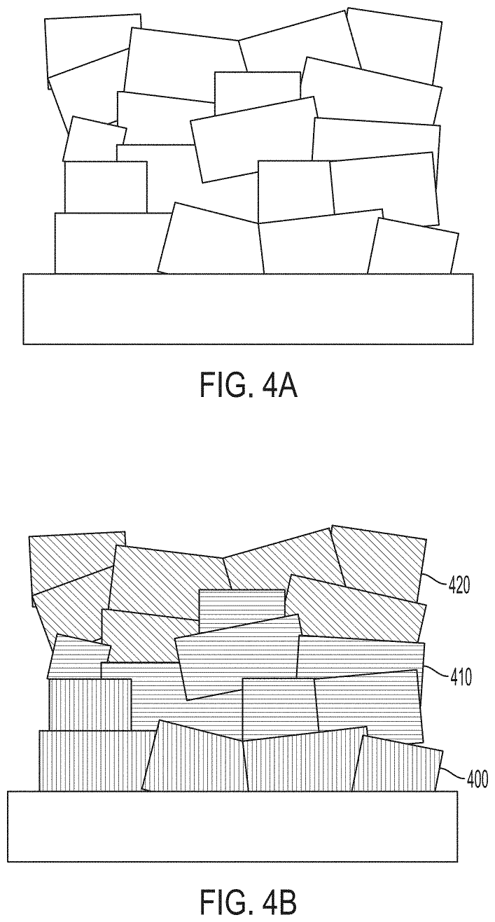

FIGS. 4A and 4B are an illustration of the anti-fouling coating formed with glass coating in which a multiple layered cellular structure has been annotated. As disclosed in FIG. 4, the cellular structure of glass crystals of the glass coating provides improved impact resistance in addition to anti-fouling effects. FIG. 4B illustrates the first layer 400, the second layer 410, and the third layer 420 of the glass coating portion of the anti-fouling coating. The gradual stacking up of the glass coating layers over multiple coating process serves to further improve the impact resistance of the glass coating.

The monomolecular coating using PFPE and glass coating provide anti-fouling function when applied to most of the surfaces of the upper jaw 220 and lower jaw 240. However, in some embodiments, increased durability of the monomolecular coating using PFPE and glass coating is desired in certain portions of the upper jaw 220 and lower jaw 240. For example, it is advantageous if the coating on the insulation pads 250 formed on the lower jaw 240 and the insulation pads (not shown) formed on the upper jaw 220 that mesh together are amplified in terms of durability so as to improve the wear resistance of the coating in these areas that an occur due to high friction imposed through grasping the tissues in between the insulation pads and applying high frequency currents. If one or both of the upper jaw 220 and the lower jaw 240 do not have insulation pads, then such durability amplified anti-fouling coatings can be applied directly to the contacting surfaces of the upper jaw 220 and/or the lower jaw 240, as such contacting surfaces would still be exposed to wear from friction through grasping the tissues in between and application of the high frequency currents.

In some embodiments, the monomolecular coating using PFPE and glass coating used for anti-fouling can have increased anti-fouling properties in certain portions of the upper jaw 220 and lower jaw 230 compared to coatings in other portions of the device. Example portions for location of an anti-fouling coating having increased anti-fouling properties include one or more of the boundary area of the lower treatment surface 242 and insulation cover 244 of the lower jaw 240, since the tissues cauterized through high frequency currents are more likely to be caught between the detents, protrusions and recesses formed in the lower jaw 240. Another example portion for location of an anti-fouling coating having increased anti-fouling properties includes the channel 260 in which the cutting blade 270 moves, since the tissues cauterized through high frequency currents are more likely to be caught in the detents, protrusions and recesses of channel 260. A still further example portion for location of an anti-fouling coating having increased anti-fouling properties includes the multiple notches 246 for securely holding grasped objects, since the tissues cauterized through high frequency currents are more likely to be caught in the structure(s) of the multiple notches 246.

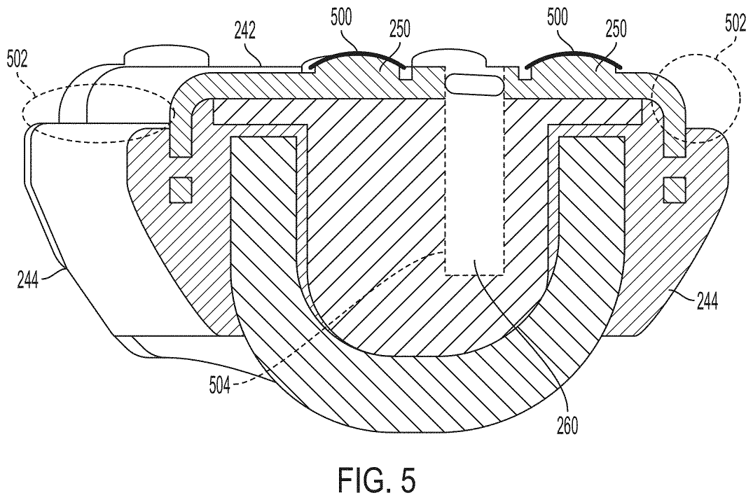

FIG. 5 illustrates a cross-sectional view of the lower jaw 240. The amplification target area(s) 500 covers the insulation pads 250, increasing the durability of the anti-fouling coating generally applied to lower jaw 240. The same amplified coating should cover the portions of upper treatment surface 222, or the insulation pads, on the upper jaw 220 that contacts the insulation pads 250 when the upper jaw 220 and lower jaw 240 close together. The amplification target area(s) 502 covers the boundary areas between the lower treatment surface 242 and insulation cover 244 of the lower jaw 240, increasing the anti-fouling property of the applied coating. Similarly, the amplification target area 504 covers the surfaces of the channel 260, increasing the anti-fouling property of the coating applied to the surfaces.

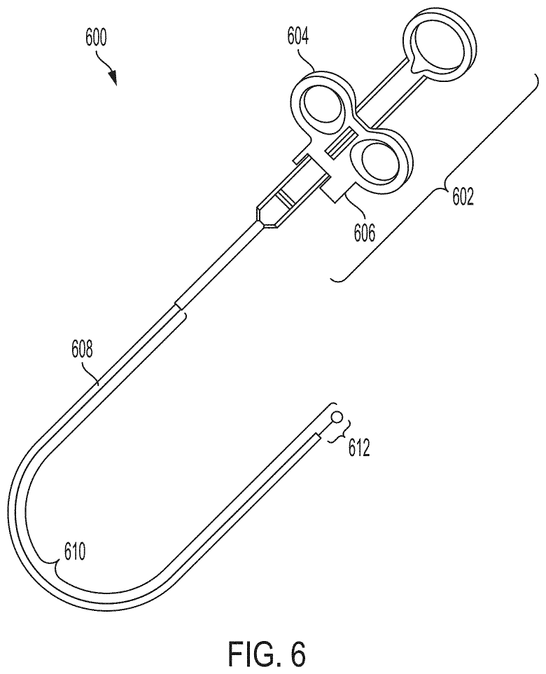

FIG. 6 is an illustration of a treatment device 600 including an operation portion 602 including a slider 604 and plug 606, tube 608, insertion tube 610, and high-frequency knife 620, which serves as monopolar electrode for dissecting tissues during the high-frequency treatment procedure.

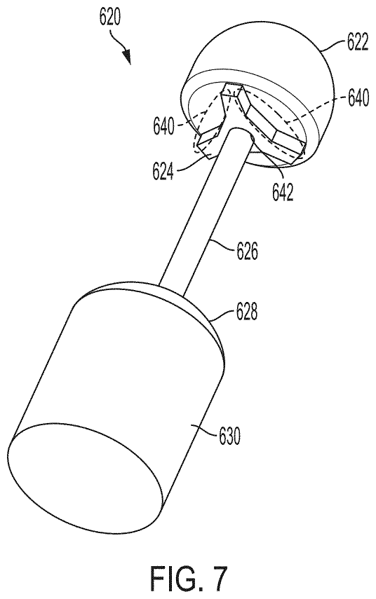

FIG. 7 is a magnified illustration of the high-frequency knife 620 including an insulation chip 622, electrode 624, dissecting knife 626, insulation cover 628, and tip portion of the insertion tube 630. High frequency currents are emitted from electrode 624 for heating the tissues to be dissected by the dissecting knife 626.

Like the bipolar treatment device 200, the surface of the monopolar treatment device 600, and especially the surfaces of the high-frequency knife 640, are coated with anti-fouling coating materials for preventing tissues cauterized through high frequency currents to stick to the treatment surfaces and other portions of the high-frequency knife 620. The coating materials that may be used for the anti-fouling coating includes glass coating and monomolecular coating using PFPE as main agent, which together with glass coating form a thin insulating film having antifouling property. The glass coating may have a cellular structure, which provides improved impact resistance in addition to anti-fouling effects.

The monomolecular coating using PFPE and glass coating used for anti-fouling also need to be amplified in certain portions of the high-frequency knife 620, amplification in terms of increased anti-fouling property. For example, the coating applied to the boundary area of the electrode 624 and insulation chip 622 (first amplification target areas including surfaces located at area 640) and boundary area of the electrode 624 and dissecting knife 626 (second amplification target area including surfaces located at area 642) need to be amplified, since the tissues cauterized through high frequency currents are more likely to be caught between the dents of the boundary areas.

The amplification increasing the durability of the anti-fouling coating of the amplification target areas, such as amplification target areas 500 and the anti-fouling coatings of the portions on the upper jaw 222 that meshes with the insulation pads 250, may be realized in two ways.

In a first way, the durability of the anti-fouling coating of the amplification target areas is increased by increasing the thickness of the anti-fouling coating. The thickness of the anti-fouling coating may be increased by making the surface of the amplification target area rougher than the other areas and performing dip-coating, which increases the amount of residual coating in the amplification target area, resulting in a thicker anti-fouling coating compared to the other areas that are not roughened. Wet coating is another procedure that may be used to thicken the anti-fouling coating in the amplification target area. A pool of liquid coating is created at the amplification target area, which increases the residual coating, resulting in a thicker coating. Re-coating (or application of multiple coating layers, such as two applications of coating layers, three applications of coating layers, etc. . . . up to ten or twenty applications of coating layers) may also be used to thicken the anti-fouling coating in the amplification target area. After performing anti-fouling coating, the portions excluding the amplification target area would be masked. The additional anti-fouling coating would be applied increasing the layer of anti-fouling coating to the amplification target area and excluding the masked portions, resulting in thicker coating in the amplification target area.

In a second way, the durability of the anti-fouling coating of the amplification target areas is increased by increasing the density of the anti-fouling coating. Since the anti-fouling coating adheres based on the polar functional groups, it is effective to deposit more polar functional groups on the amplification target areas in order to increase the density of the anti-fouling coating. In order to deposit more polar functional groups, the amplification target area may go through procedures such as (i) corona discharge, (ii) plasma treatment, or (iii) application of ultraviolet light. By limiting the type of polar functional groups to hydroxyl groups, (iv) boiling may also be an effective procedure to increase the density of the anti-fouling coating at the amplification target area. Furthermore, since hydroxyl groups are often deposited on a glass surface, (v) adding glass coating on the amplification target area in advance to application of the procedures (i) to (iv) and application of the anti-fouling coating may also be effective to increase the density of the anti-fouling coating and thereby increase its durability.

The amplification increasing the anti-fouling property of the anti-fouling coating of the amplification target areas, such as amplification target areas 502, 504, and the detents, protrusions and recesses associated with notches 246 for bipolar electrodes and amplification target areas 640 and 642 for monopolar electrodes may be realized in two ways.

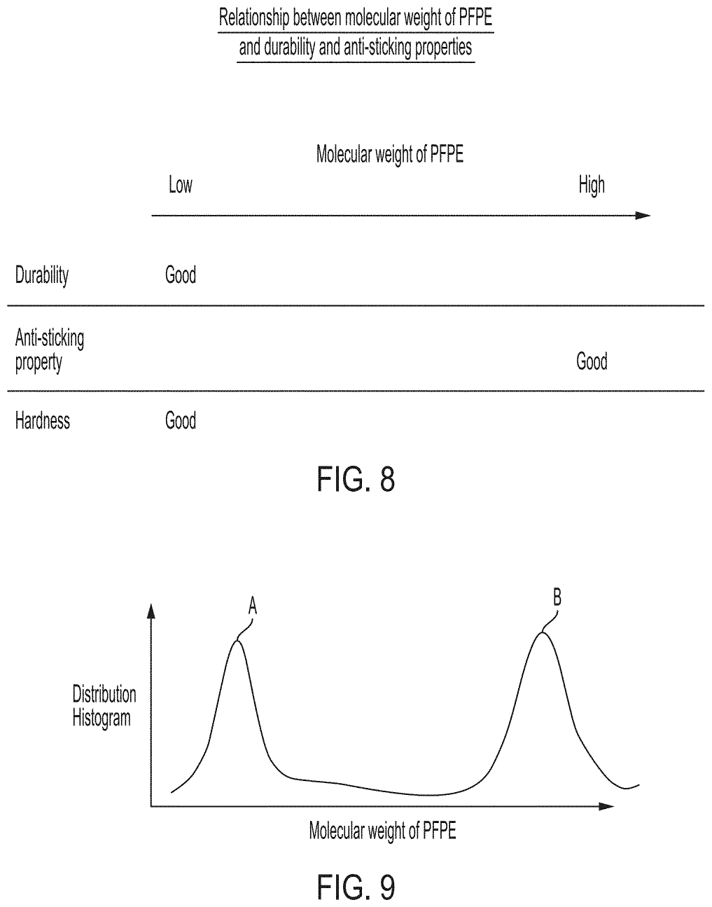

In a first way, the anti-fouling property of the anti-fouling coating of the amplification target areas is increased by applying a coating with PFPE having higher molecular weight compared with the molecular weight of the anti-fouling coating used in the non-amplification target areas. As disclosed in FIG. 8, the anti-fouling property of the anti-fouling coating increases as the molecular weight of the PFPE increases. Thus, after applying anti-fouling coating to the entire device using PFPE having lower molecular weight compared to the molecular weight of the anti-fouling coating used in the amplification target areas, blasting or other removal procedure would be applied to the amplification target area requiring enhanced anti-fouling property to peel off the anti-fouling coating with PFPE having lower molecular weight. Then, anti-fouling coating using PFPE with higher molecular weight would be applied, which would only stick to the amplification target area where the blasting procedure had been performed, leaving anti-fouling coating using PFPE with higher molecular weight to reside at the amplification target area. As also shown in FIG. 8, the low molecular weight of PFPE is beneficial for the durability and hardness properties of the anti-fouling coating, serving to keep the durability and hardness properties in the portions other than the amplification target area that requires enhanced anti-fouling properties.

In a second way, the anti-fouling property of the anti-fouling coating of the amplification target areas is increased by increasing the density of the anti-fouling coating. Since the anti-fouling coating adheres based on the polar functional groups, it is effective to deposit more polar functional groups on the amplification target areas (i.e. a high area density of polar functional groups) in order to increase the density of the anti-fouling coating. In order to deposit more polar functional groups, the amplification target area may go through procedures such as (i) corona discharge, (ii) plasma treatment, or (iii) application of ultraviolet light. By limiting the type of polar functional groups to hydroxyl groups, (iv) boiling may also be an effective procedure to increase the density of the anti-fouling coating at the amplification target area. Furthermore, since hydroxyl groups are often deposited on a glass surface, (v) adding glass coating on the amplification target area in advance to application of the procedures (i) to (iv) and application of the anti-fouling coating may also be effective to increase the density of the anti-fouling coating and thereby increase its anti-fouling property.

FIG. 9 is a chart illustrating the distribution histogram of an anti-fouling coating including PFPE with varying molecular weights (“blended coating”). As shown in FIG. 9, the distribution histogram of the blended coating includes peak A indicating concentration of PFPE with low molecular weight and peak B indicating concentration of PFPEs with high molecular weight. The combination of the PFPEs with varying molecular weights and thus varying characteristics at a calculated proportion would provide the anti-fouling coating the desired property in accordance to the required specifications for the anti-fouling coating.

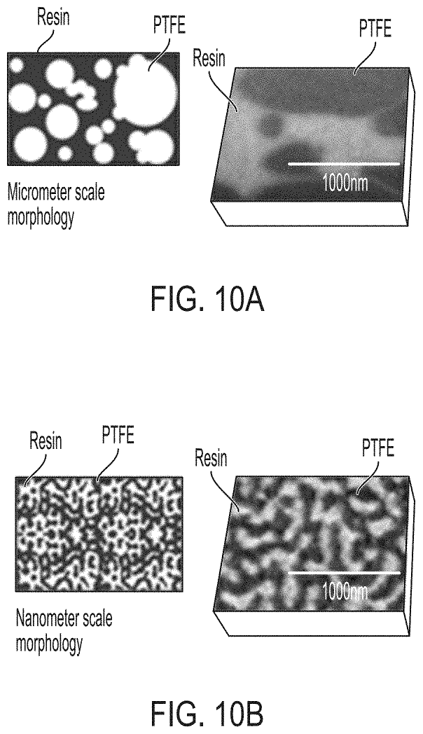

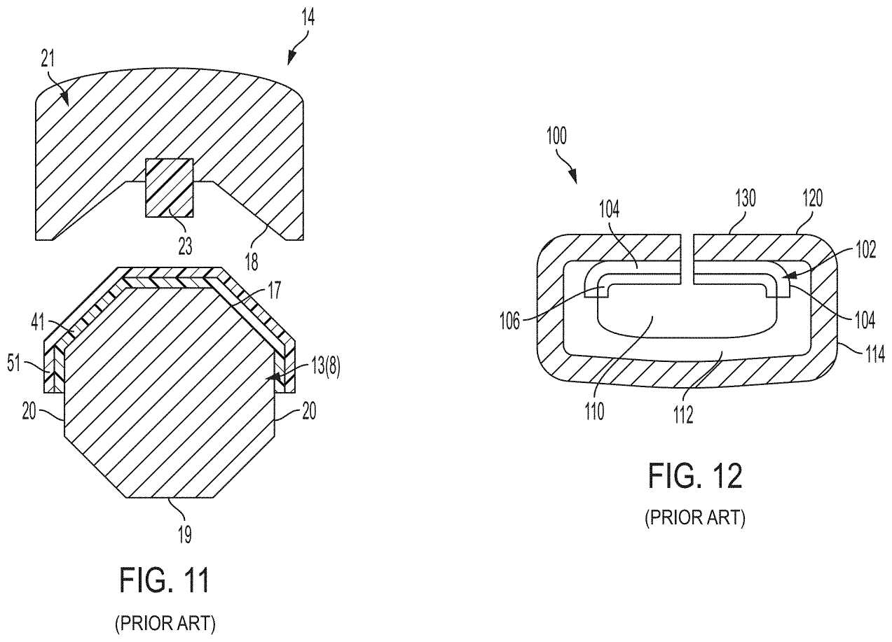

FIG. 10A illustrates a micrometer scale morphology of composite material of PTFE and resin that are used for insulating covers 224 and 244. As shown in FIG. 10A, the insulating composite material of PTFE and resin includes unevenness in the size and distribution of the PTFE within the resin substrate. When a monomolecular coating is applied to the surface of the composite material, a solid monomolecular layer will be formed in the area where the resin is present and a thin and fragile layer would be formed where the PTFE is present, resulting in a reticulated coating which has negative effects to anti-fouling property. Thus, not applying the anti-fouling coating to the insulating covers 224 and 244 is desirable.

A composite material of PTFE and resin with a nanometer scale morphology as shown in FIG. 10B would not have the above mentioned unevenness but would be expensive to use. Moreover, since the nanometer scale morphology composite material of PTFE and resin itself would have sufficient anti-fouling property, applying anti-fouling coating to composite material of PTFE and resin having nanometer scale morphology would not be necessary. Thus, in this instance, not applying the anti-fouling coating to the insulating covers 224 and 244 is desirable.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A treatment device, comprising:
a body including a connection configured to connect to a power source to supply power for conducting a high-frequency treatment with the treatment device; and
a longitudinally extending shaft having a proximal end connected to the body and a distal end defining a treatment end,
wherein the treatment end includes a first bipolar electrode and a second bipolar electrode for conducting high-frequency currents including a conductive treatment surface and an insulating cover,
wherein the treatment end is coated with an anti-fouling coating including an insulating substance, and
wherein the treatment end is coated with a durability amplified coating or an anti-fouling amplified coating.

2. The treatment device according to claim 1, wherein the conductive treatment surface of the first bipolar electrode includes an insulation pad that is coated with the durability amplified coating.

3. The treatment device according to claim 2, wherein the conductive treatment surface of the second bipolar electrode includes an insulation pad that is coated with the durability amplified coating.

4. The treatment device according to claim 1, wherein the conductive treatment surface of the first bipolar electrode includes an insulation pad, and wherein the conductive treatment surface of the second bipolar electrode that contacts the insulation pad of the first bipolar electrode when the first and second bipolar electrodes are closed together is coated with the durability amplified coating.

5. The treatment device according to claim 4, wherein the first bipolar electrode includes a cutting blade channel that is coated with the anti-fouling coating.

6. The treatment device according to claim 1, wherein the anti-fouling coating includes glass.

7. The treatment device according to claim 3, wherein the thickness of the anti-fouling coating is 1 μm or less.

8. The treatment device according to claim 1, wherein a surface of the treatment end on which the durability amplified coating is applied is rougher as compared to a surface of the treatment end on which the anti-fouling coating is applied.

9. The treatment device according to claim 1, wherein a molecular weight of the insulating substance in the anti-fouling coating is different as compared to the durability amplified coating or the anti-fouling coating.

10. The treatment device according to claim 1, wherein a molecular weight of the insulating substance in the anti-fouling coating is lower than a molecular weight of the insulating substance in the anti-fouling amplified coating.

11. The treatment device according to claim 1, wherein a base of the anti-fouling amplified coating is configured to have a higher density of polar functional groups as compared to the anti-fouling coating.

12. The treatment device according to claim 1, wherein a frequency of bonding between the insulating substance and a base material on a surface of the treatment end is higher at the durability amplified coating as compared to the anti-fouling coating.

13. The treatment device according to claim 1, wherein a frequency of bonding between the insulating substance and a base material on a surface of the treatment end is higher at the anti-fouling amplified coating as compared to the anti-fouling coating.

14. The treatment device according to claim 2, wherein the anti-fouling amplified coating is bonded to the hydroxyl group formed on the glass coating base.

15. The treatment device according to claim 1, wherein a glass coating base is formed between a surface of the treatment end and the durability amplified coating.

16. The treatment device according to claim 2, wherein a glass coating base is formed between a surface of the treatment end and the anti-fouling amplified coating.

17. The treatment device according to claim 1, wherein the insulating cover includes an antifouling substance that is less likely to bond with the anti-fouling coating as compared to the other substances of the insulating cover.

18. The treatment device according to claim 2, wherein the insulating cover includes a portion not coated with anti-fouling coating.

19. The treatment device according to claim 15, wherein the insulating cover placed opposite to the conductive treatment surface includes a portion not coated with the anti-fouling coating.

20. The treatment device according to claim 16, wherein the insulating cover placed opposite to the conductive treatment surface includes a portion not coated with the anti-fouling coating.
